(19) United States
(12) Patent Application Publication
(10) Pub. No.: US 2025/0178997 A1
(43) Pub. Date: Jun. 5, 2025

(54) ORGANIC COMPOUNDS

(71) Applicant: GIVAUDAN SA, VERNIER (CH)

(72) Inventors: Marc LINIGER, Kemptthal (CH); Sandro DOSSENBACH, Kemptthal (CH); Lucas MACEDO DE ABREU HORTENCIO, Kemptthal (CH); Martin Alan LOVCHIK, Kemptthal (CH)

(21) Appl. No.: 18/842,101

(22) PCT Filed: Mar. 9, 2023
(86) PCT No.: PCT/EP2023/055969

§ 371 Date: Aug. 28, 2024

(30) Foreign Application Priority Data
Mar. 10, 2022 (GB) 2203328.6

(51) Int. Cl. C07C 49/417; A23L 27/00; A23L 27/20; C07C 49/537

(52) U.S. Cl. CPC C07C 49/417; A23L 27/203; A23L 27/88; C07C 49/537; C07C 2601/02; C07C 2601/10

(57) ABSTRACT

Disclosed are 3-alkylcyclopent-2-en-1-one and 3-alkylcyclopentan-1-one derivatives of formula (I) possessing powerful spicy woody notes.

### TECHNICAL FIELD

The present invention is concerned with flavor and fragrance ingredients, and to compositions containing them that are useful in flavor and fragrance applications. It furthermore relates to a method of making them. In particular the present invention is concerned with 3-alkylcyclopent-2-en-1-one derivatives of formula (I) possessing powerful spicy woody notes.

### BACKGROUND

In the flavor and fragrance industry, perfumers and flavorists are continually looking for new compounds possessing unique olfactory properties. For example, there is a need for ingredients that are suitable for the flavor and fragrance industry possessing spicy, woody notes. Woody and spicy notes are fundamental olfactive families as for instance jasmine and floral. They can not only be a key character of a perfume, but also support and complement perfume accords in general.

Surprisingly inventors found a new class of ingredients of formula (I) as defined herein below possessing the highly sought-after spicy woody notes. This new class of ingredients supports and nurtures, in particular spicy woody fragrance accords.

### SUMMARY

In accordance with a first aspect of the present invention there is provided a compound of formula (I) in the form of any one of its stereoisomers or mixture thereof

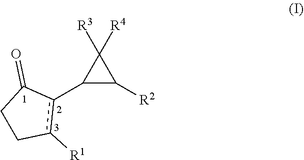

wherein
R¹ is selected from C₁-C₃ alkyl (e.g. ethyl, n-propyl, iso-propyl);
R² is a hydrocarbon residue comprising up to 10 carbon atoms,
R³ and R⁴ are independently selected from hydrogen and methyl with the proviso that R³ or
R⁴ is methyl; and the dotted line together with the carbon-carbon bond forms a single bond or a double bond.

In accordance with a second aspect of the present invention there is provided a fragranced article comprising as odorant a compound of formula (I) of the first aspect.

In accordance with a third aspect of the present invention there is provided a method of improving, enhancing and/or modifying a consumer product base or consumable product base by means of adding thereto an olfactory acceptable amount of a compound for formula (I) of the first aspect.

In accordance with a fourth aspect of the present invention there is provided a flavored product comprising a compound of formula (I) of the first aspect.

In accordance with a fifth aspect of the present invention there is provided a fragrance or flavor composition comprising a compound of formula (I) of the first aspect.

In accordance with a sixth aspect of the present invention there is provided the use as flavor or fragrance of a compound of formula (I) of the first aspect.

Certain embodiments of any aspect of the present invention may provide one or more of the following advantages:
provision of novel flavor and fragrance compounds with spicy, woody notes,
provision of a highly performing compound with low odor threshold.

The details, examples and preferences provided in relation to any particular one or more of the stated aspects of the present invention will be further described herein and apply equally to all aspects of the present invention. Any combination of the embodiments, examples and preferences described herein in all possible variations thereof is encompassed by the present invention unless otherwise indicated herein, or otherwise clearly contradicted by context.

### DETAILED DESCRIPTION

The present invention is based on the surprising finding that 3-alkylcyclopent-2-en-1-one derivatives as defined herein possessing powerful spicy woody notes.

In a first aspect of the invention, there is provided a compound of formula (I) in the form of any one of its stereoisomers or mixture thereof

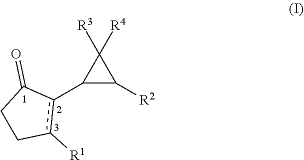

wherein
R¹ is selected from C₁-C₃ alkyl (e.g. ethyl, n-propyl, iso-propyl);
R² is a hydrocarbon residue comprising up to 10 carbon atoms,
R³ and R⁴ are independently selected from hydrogen and methyl with the proviso that R³ or R⁴ is methyl; and
the dotted line together with the carbon-carbon bond forms a single bond or a double bond.

In one particular embodiment there is provided a compound of formula (II) in the form of any one of its stereoisomers or mixture thereof

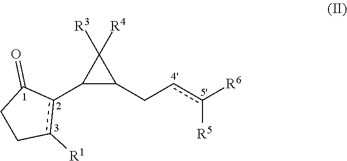

wherein
R¹ is selected from C₁-C₃ alkyl (e.g. ethyl, n-propyl, iso-propyl);
R⁶ is selected from hydrogen, and C₁-C₃ alkyl (e.g. ethyl, n-propyl, iso-propyl);
R³ and R⁴ are independently selected from hydrogen and methyl with the proviso that R³ or R⁴ is methyl; and
R⁵ is selected from hydrogen, and C₁-C₃ alkyl (e.g. ethyl, n-propyl, iso-propyl); and
the each of the dotted lines together with the carbon-carbon bond forms a single bond or a double bond.

In another particular embodiment the 3-alkylcyclopent-2-en-1-one derivatives is a compound of formula (III) in the form of any one of its stereoisomers or mixture thereof

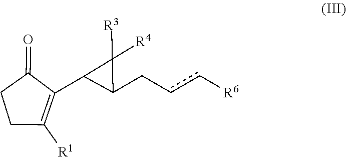

wherein
R¹ is methyl or ethyl, and
R⁶ is methyl or ethyl,
R³ and R⁴ are independently selected from hydrogen and methyl, with the proviso that R³ or R⁴ is methyl, and
the dotted line together with the carbon-carbon bond forms a single bond or a double bond.

In a further particular embodiment the 3-alkylcyclopent-2-en-1-one derivatives is a compound of formula (IV) in the form of any one of its stereoisomers or mixture thereof

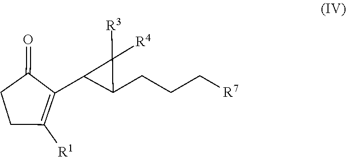

wherein
R¹ is methyl or ethyl, and
R⁷ is methyl or ethyl,
R³ and R⁴ are independently selected from hydrogen and methyl.

In one particular embodiment the compound of formula (I) (which encompasses the compounds of formula (II), (III) and (IV)) is a compound wherein R³ and R⁴ are methyl.

In one particular embodiment the compound of formula (I) (which encompasses the compounds of formula (II), (III) and (IV)) is a compound wherein R¹, R³ and R⁴ are methyl.

In another particular embodiment the compounds of formula (I) is selected from the group consisting of 2-(3-butyl-2,2-dimethylcyclopropyl)-3-methylcyclopent-2-en-1-one, (3-(but-2-en-1-yl)-2,2-dimethylcyclopropyl)-3-methylcyclopent-2-en-1-one, 2-(2,2-dimethyl-3-(pent-2-en-1-yl)cyclopropyl)-3-methylcyclopent-2-en-1-one, 2-(2,2-dimethyl-3-pentylcyclopropyl)-3-methylcyclopent-2-en-1-one, 2-(3-butyl-2,2-dimethylcyclopropyl)-3-propylcyclopent-2-en-1-one, 2-(3-butyl-2,2-dimethylcyclopropyl)-3-ethylcyclopent-2-en-1-one, 2-(3-(but-2-en-1-yl)-2,2-dimethylcyclopropyl)-3-ethylcyclopent-2-en-1-one, 2-(3-(hex-2-en-1-yl)-2,2-dimethylcyclopropyl)-3-methylcyclopent-2-en-1-one, 2-(3-hexyl-2,2-dimethylcyclopropyl)-3-methylcyclopent-2-en-1-one. 3-methyl-2-(2,2,3-trimethylcyclopropyl)cyclopent-2-en-1-one, 2-(3-ethyl-2,2-dimethylcyclopropyl)-3-methylcyclopent-2-en-1-one, 2-(2,2-dimethyl-3-propylcyclopropyl)-3-methylcyclopent-2-en-1-one, 2-(3-allyl-2,2-dimethylcyclopropyl)-3-methylcyclopent-2-en-1-one, and 2-(3-(but-2-en-1-yl)-2,2-dimethylcyclopropyl)-3-methylcyclopent-2-en-1-one.

The compounds according to the present invention comprise one or more chiral centres and as such may exist as a mixture of stereoisomers, or they may be resolved as isomerically pure forms. Resolving stereoisomers adds to the complexity of manufacture and purification of these compounds and so it is preferred to use the compounds as mixtures of their stereoisomers simply for economic reasons. However, if it is desired to prepare individual stereoisomers, this may be achieved according to methodology known in the art, e.g. preparative HPLC and GC or by stereoselective synthesis.

As one specific example one may cite 2-(3-butyl-2,2-dimethylcyclopropyl)-3-methylcyclopent-2-en-1-one comprising two stereogenic centers and thus may exist as a mixture of up to four stereoisomers ((Va) to (Vd) as shown below), in its diastereomerically pure or even enantiomerically pure forms. Some of the individual isomers have different odor qualities, both in terms of performance as well as olfactory properties.

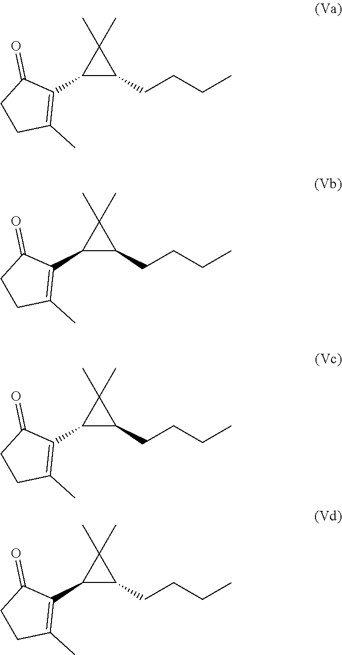

For example, one may cite 2-((1S,3R)-3-butyl-2,2-dimethylcyclopropyl)-3-methylcyclopent-2-en-1-one (Va) possessing in addition to its overall spicy woody odor notes also green, orris, rooty and vetiver facets. 2-((1S,3R)-3-butyl-2,2-dimethylcyclopropyl)-3-methylcyclopent-2-en-1-one has remarkable low odor threshold concentration (below 1 pg) which is about 50 000 times lower than 2-((1R,3S)-3-butyl-2,2-dimethylcyclopropyl)-3-methylcyclopent-2-en-1-one (Vb), and about 5000 times lower than rel-2-((1S,3S)-3-butyl-2,2-dimethylcyclopropyl)-3-methylcyclopent-2-en-1-one.

As a further example one may cite 2-((1S,3R)-3-(but-2-en-1-yl)-2,2-dimethylcyclopropyl)-3-methylcyclopent-2-en-1-one also having a remarkable low odor threshold, which is about 6 times higher than 2-((1S,3R)-3-butyl-2,2-dimethylcyclopropyl)-3-methylcyclopent-2-en-1-one (Va).

Thus there is provided in another embodiment a compound of formula (I) (which encompasses compounds of formula (II) and (III)) wherein the relative configuration is in 1S, 3R, as shown by formula (Ia)

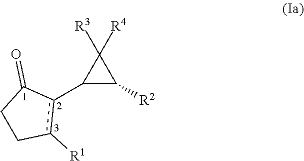

wherein R¹, R², R³ and R⁴ have the same meaning as given for compounds of formula (I).

There is provided in a further embodiment a compound of formula (II) (which encompasses compounds of formula (III) and (IV)) wherein the relative configuration is in 1S, 3R, as shown by formula (IIa)

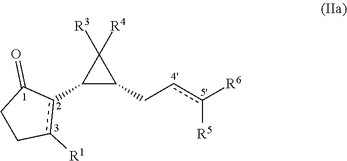

wherein R¹, R³, R⁴, R⁵ and R⁶ have the same meaning as given for compounds of formula (II).

As used herein, “hydrocarbon residue” means a monovalent residue comprising 1 to 10 carbon atoms (for example 2 to 9 C atoms, 3 to 8 C atoms, 4 to 7 C atoms, 4 to 6 C atoms), which includes C₁-C₁₀ alkyl (linear or branched) such as methyl, ethyl, n-propyl, isopropyl, n-butyl, tert-butyl, isobutyl, sec-butyl, isopentyl, 2-methylbutyl, n-pentyl, 4-methylpentyl, n-hexyl, n-heptyl, and n-octyl, and C₂-C₁₀ alkenyl (linear or branched) comprising at least one double bond (e.g. up to 3 double bonds) such as 3-methyl-but-2-en-1-yl, 2-methylprop-1-en-1-yl, prop-1-en-1-yl, vinyl, but-1-en-1-yl, but-2-en-1-yl, but-3-en-1-yl, pent-1-en-1-yl, buta-1,3-dien-1-yl, and penta-1,3-dien-1-yl.

The compounds of formula (I) (which encompasses compounds of formula (II), (III) and (IV)) may be used alone, as isomeric mixture thereof, or in combination with known odorant molecules selected from the extensive range of natural products, and synthetic molecules currently available, such as essential oils, alcohols, aldehydes and ketones, ethers and acetals, esters and lactones, macrocycles and heterocycles, and/or in admixture with one or more ingredients or excipients conventionally used in conjunction with odorants in fragrance compositions, for example, carrier materials, and other auxiliary agents commonly used in the art.

As used herein, “carrier material” means a material which is practically neutral from a odorant point of view, i.e. a material that does not significantly alter the organoleptic properties of odorants.

The term “auxiliary agent” refers to ingredients that might be employed in a fragrance composition for reasons not specifically related to the olfactive performance of said composition. For example, an auxiliary agent may be an ingredient that acts as an aid to processing a fragrance ingredient or ingredients, or a composition containing said ingredient(s), or it may improve handling or storage of a fragrance ingredient or composition containing same. It might also be an ingredient that provides additional benefits such as imparting color or texture. It might also be an ingredient that imparts light resistance or chemical stability to one or more ingredients contained in a fragrance composition. A detailed description of the nature and type of adjuvants commonly used in fragrance compositions containing same cannot be exhaustive, but it has to be mentioned that said ingredients are well known to a person skilled in the art.

As used herein, ‘fragrance composition’ means any composition comprising a compound of formula (I) (which encompasses compounds of formula (II) and (III)), or a mixture thereof and a base material, e.g. a diluent conventionally used in conjunction with odorants, such as diethyl phthalate (DEP), dipropylene glycol (DPG), isopropyl myristate (IPM), pentane-1,2-diol, triethyl citrate (TEC) and alcohol (e.g. ethanol). Optionally, the composition may comprise an anti-oxidant adjuvant. Said anti-oxidant may be selected from Tinogard® TT (BASF), Tinogard® Q (BASF), Tocopherol (including its isomers, CAS 59-02-9; 364-49-8; 18920-62-2; 121854-78-2), 2,6-bis(1,1-dimethylethyl)-4-methylphenol (BHT, CAS 128-37-0) and related phenols, hydroquinones (CAS 121-31-9).

The following list comprises examples of known odorant molecules, which may be combined with a compound of formula (I) (which encompasses compounds of formula (Ia)), or a mixture thereof:
essential oils and extracts, e.g. castoreum, costus root oil, oak moss absolute, geranium oil, tree moss absolute, basil oil, fruit oils, such as bergamot oil and mandarine oil, myrtle oil, palmarose oil, patchouli oil, petitgrain oil, jasmine oil, rose oil, sandalwood oil, wormwood oil, gurjun balsam oil, lavender oil and/or ylang-ylang oil;
alcohols, e.g. cinnamic alcohol ((E)-3-phenylprop-2-en-1-ol); cis-3-hexenol ((Z)-hex-3-en-1-ol); citronellol (3,7-dimethyloct-6-en-1-ol); dihydro myrcenol (2,6-dimethyloct-7-en-2-ol); Ebanol™ ((E)-3-methyl-5-(2,2,3-trimethylcyclopent-3-en-1-yl)pent-4-en-2-ol); eugenol (4-allyl-2-methoxyphenol); ethyl linalool ((E)-3,7-dimethylnona-1,6-dien-3-ol); farnesol ((2E,6Z)-3,7,11-trimethyldodeca-2,6,10-trien-1-ol); geraniol ((E)-3,7-dimethylocta-2,6-dien-1-ol); Super Muguet™ ((E)-6-ethyl-3-methyloct-6-en-1-ol); linalool (3,7-dimethylocta-1,6-dien-3-ol); menthol (2-isopropyl-5-methylcyclohexanol); Nerol (3,7-dimethyl-2,6-octadien-1-ol); phenyl ethyl alcohol (2-phenylethanol); Rhodinol™ (3,7-dimethyloct-6-en-1-ol); Sandalore™ (3-methyl-5-(2,2,3-trimethylcyclopent-3-en-1-yl)pentan-2-ol); terpineol (2-(4-methylcyclohex-3-en-1-yl)propan-2-ol); or Timberol™ (1-(2,2,6-trimethylcyclohexyl)hexan-3-ol); 2,4,7-trimethylocta-2,6-dien-1-ol, and/or [1-methyl-2(5-methylhex-4-en-2-yl)cyclopropyl]-methanol;
aldehydes and ketones, e.g. anisaldehyde (4-methoxybenzaldehyde); alpha amyl cinnamic aldehyde (2-benzylideneheptanal); Georgywood™ (1-(1,2,8,8-tetramethyl-1,2,3,4,5,6,7,8-octahydronaphthalen-2-yl)ethanone); Hydroxycitronellal (7-hydroxy-3,7-dimethyloctanal); Iso E Super® (1-(2,3,8,8-tetramethyl-1,2,3,4,5,6,7,8-octahydronaphthalen-2-yl)ethanone); Isoraldeine® ((E)-3-methyl-4-(2,6,6-trimethylcyclohex-2-en-1-yl)but-3-en-2-one); Hedione™ (methyl 3-oxo-2-pentylcyclopentaneacetate); 3-(4-isobutyl-2-methylphenyl)propanal; maltol; methyl cedryl ketone; methylionone; verbenone; and/or vanillin;
ether and acetals, e.g. Ambrox® (3a,6,6,9a-tetramethyl-2,4,5,5a,7,8,9,9b-octahydro-1H-benzo[e][1]benzofuran); geranyl methyl ether ((2E)-1-methoxy-3,7-dimethylocta-2,6-diene); and/or Spirambrene® (2′,2′,3,7,7-pentamethylspiro[bicyclo[4.1.0]heptane-2,5′-[1,3]dioxane]);
esters and lactones, e.g. benzyl acetate; cedryl acetate ((1S,6R,8aR)-1,4,4,6-tetramethyloctahydro-1H-5,8a-methanoazulen-6-yl acetate); delta-decalactone (6-pentyltetrahydro-2H-pyran-2-one); Helvetolide® (2-(1-(3,3-dimethylcyclohexyl)ethoxy)-2-methylpropyl propionate); delta-undecalactone (5-heptyloxolan-2-one); and/or vetiveryl acetate ((4,8-dimethyl-2-propan-2-ylidene-3,3a,4,5,6,8a-hexahydro-1H-azulen-6-yl) acetate);
macrocycles, e.g. Ambrettolide ((Z)-oxacycloheptadec-10-en-2-one); ethylene brassylate (1,4-dioxacycloheptadecane-5,17-dione); and/or Exaltolide® (16-oxacyclohexadecan-1-one); and
heterocycles, e.g. isobutylquinoline (2-isobutylquinoline).

Thus there is provided in a further aspect of the invention a fragrance composition comprising a compound of formula (I) (which encompasses compounds of formula (II), (III) and (IV)).

The compounds of formula (I) (which encompasses compounds of formula (II), (III) and (IV) may be used in a broad range of fragranced articles, e.g. in any field of fine and functional perfumery, such as perfumes, air care products, household products, laundry products, body care products and cosmetics. The compound can be employed in widely varying amounts, depending upon the specific article and on the nature and quantity of other odorant ingredients. The proportion is typically from 0.00001 to 3 weight percent of the article. In one embodiment, the compound may be employed in a fabric softener in an amount from 0.0001 to 1 weight percent (e.g. 0.001 to 0.1 including 0.05 and 0.03 weight %). In another embodiment, the compound may be used in fine perfumery in amounts from 0.00001 to 30 weight percent (e.g. up to about 10 or up to 20 weight percent), more preferably between 0.01 and 5 weight percent (e.g. 0.01 to 0.1 weight percent). However, these values are given only by way of example, since the experienced perfumer may also achieve effects or may create novel accords with lower or higher concentrations.

In another embodiment the compounds of formula (I) (which encompasses compounds of formula (II), (III) and (IV)) may be used in a broad range of flavored products including alcoholic and non-alcoholic beverages, e.g. teas, frozen dairy desserts, confectionary and bakery goods, gelatines, puddings, meat and meat products, and tobacco. The compounds of formula (I) (which encompasses compounds of formula (II), (III) and (IV)) may also be used as for flavour compositions, for example, in grape flavours, plum flavours, dried fruit flavours, red berry flavours such as raspberry, blackberry or gooseberry flavours. They may also be used as flavour enhancers, for example, in pepper, ginger, basil, rosemary, cardamom, nutmeg, cinnamon, peppermint, grape (such as Shiraz grape), juniper and grapefruit flavours.

When used in flavoured products, the compounds of the present invention may be present in amounts ranging from 0.01 ppb (10⁻¹¹) to 10 ppb (10⁻⁸) by weight based on the consumable product, more preferably from 0.1 ppb (10⁻¹⁰) to 1 ppb (10⁻⁹) by weight. However, these values should not be limiting on the present invention, since the experienced flavourist may also achieve effects or may create novel accords with lower or higher concentrations.

The compounds of formula (I) (which encompasses compounds of formula (II), (III) and (IV)) may be employed in a consumer product base simply by directly mixing the compound, or a fragrance composition comprising a compound of formula (I) (which encompasses compounds of formula (II), (III) and (IV)), or a mixture thereof, with the consumer product base, or it may, in an earlier step, be entrapped with an entrapment material, for example, polymers, capsules, microcapsules and nanocapsules, liposomes, film formers, absorbents such as carbon or zeolites, cyclic oligosaccharides and mixtures thereof, and then mixed with the consumer product base.

Thus, the invention additionally provides a method of manufacturing a fragranced article or flavored product, comprising the incorporation a compound of formula (I) (which encompasses compounds of formula (II), (III) and (IV)), or a mixture thereof as a flavor and/or fragrance ingredient, either by directly admixing to the consumer product base or by admixing a fragrance/flavor composition comprising a compound of formula (I) (which encompasses compounds of formula (II), (III) and (IV)), or a mixture thereof, which may then be mixed with a consumer product base, using conventional techniques and methods. Through the addition of an olfactory acceptable amount of a compound of formula (I) (which encompasses compounds of formula (II), (III) and (IV)), or a mixture thereof the odor notes of a consumer product base will be improved, enhanced, or modified.

Thus, the invention furthermore provides a method for improving, enhancing or modifying a consumer product base by means of the addition thereto of an olfactorily acceptable amount of a compound of formula (I) (which encompasses compounds of formula (II), (III) and (IV)), or a mixture thereof.

There is provided in a further aspect of the present invention a fragranced article comprising:
a) a compound of formula (I)

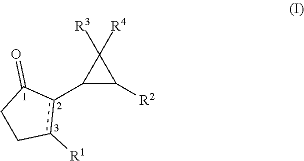

wherein
   R¹ is selected from C₁-C₃ alkyl (e.g. ethyl, n-propyl, iso-propyl);
   R² is a hydrocarbon residue comprising up to 10 carbon atoms,
   R³ and R⁴ are independently selected from hydrogen and methyl with the proviso that R³ or R⁴ is methyl; and
   the dotted line together with the carbon-carbon bond forms a single bond or a double bond; and
b) a consumer product base

As used herein, ‘consumer product base’ means a composition for use as a consumer product to fulfill specific actions, such as cleaning, softening, and caring or the like. Examples of such products include fine perfumery, e.g. perfume and eau de toilette; fabric care, household products and personal care products such as cosmetics, laundry care detergents, rinse conditioner, personal cleansing composition, detergent for dishwasher, surface cleaner; laundry products, e.g. softener, bleach, detergent; body-care products, e.g. shampoo, shower gel; air care products (includes products that contain preferably volatile and usually pleasant-smelling compounds which advantageously can even in very small amounts mask unpleasant odors). Air fresheners for living areas contain, in particular, natural and synthetic essential oils such as pine needle oils, citrus oil, eucalyptus oil, lavender oil, and the like, in amounts for example of up to 50% by weight. As aerosols they tend to contain smaller amounts of such essential oils, by way of example less than 5% or less than 2% by weight, but additionally include compounds such as acetaldehyde (in particular, <0.5% by weight), isopropyl alcohol (in particular, <5% by weight), mineral oil (in particular, <5% by weight), and propellants.

Cosmetic products include:
(a) cosmetic skincare products, especially bath products, skin washing and cleansing products, skincare products, eye makeup, lip care products, nail care products, intimate care products, foot care products;
(b) cosmetic products with specific effects, especially sunscreens, tanning products, de-pigmenting products, deodorants, antiperspirants, hair removers, and shaving products;
(c) cosmetic dental-care products, especially dental and oral care products, tooth care products, cleaners for dental prostheses, adhesives for dental prostheses; and
(d) cosmetic hair care products, especially hair shampoos, hair care products, hair setting products, hair-shaping products, and hair coloring products.

This list of products is given by way of illustration, and is not to be regarded as being in any way limiting.

In one particular embodiment the consumer product base is selected form fine perfumery, and personal care products, including deodorants, hair care products, soaps, and the like.

In a further particular embodiment the consumer product base is selected from fabric care products, including fabric softener, and home care products, including air fresheners, dish washers and the like.

There is provided in a further aspect a flavored product comprising:
a) a compound of formula (I)

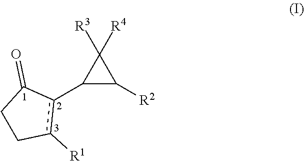

wherein
   R¹ is selected from C₁-C₃ alkyl (e.g. ethyl, n-propyl, iso-propyl);
   R² is a hydrocarbon residue comprising up to 10 carbon atoms,
   R³ and R⁴ are independently selected from hydrogen and methyl with the proviso that R³ or R⁴ is methyl; and
   the dotted line together with the carbon-carbon bond forms a single bond or a double bond; and
b) a consumable product base.

As used herein, ‘consumable product base’ means a composition for use as consumable product, such as foodstuff and beverages, or personal care products that are intended to be introduced into the oral cavity of a human or animal and remain there for a certain period of time before being ingested or removed from the mouth. Such products include composition in there processed, partially processed or unprocessed state.

The compounds of formula (I) (which encompasses compounds of formula (II), (III)) and (IV)) wherein R² is selected from alky and alkenyl may, for example, be synthesized by an ozonolysis of delta-3-carene followed by Wittig reaction, hydrogenation, alkylation and ring closure or by a cyclopropanation reaction of an alkene or by a Stetter reaction of an aldehyde with an alpha,beta-unsaturated ketone followed by ring closure.

The compounds of formula (I) (which encompasses compounds of formula (II), (III)) and (IV)) wherein R² is selected from alkoxy may, for example, be synthesized by an ozonolysis of delta-3-carene followed by etherification, followed by alkylation and ring closure.

The invention is now further described with reference to the following non-limiting examples. These examples are for the purpose of illustration only and it is understood that variations and modifications can be made by one skilled in the art.

### Example 1: 2-((1S,3R)-3-butyl-2,2-dimethylcyclopropyl)-3-methylcyclopent-2-en-1-one

### a) Synthesis of 2-((1R,3S)-2,2-dimethyl-3-(2-oxopropyl)cyclopropyl)acetaldehyde

A solution of (1S,6R)-3,7,7-trimethylbicyclo[4.1.0]hept-3-ene ((+)-3-carene, [α]_{D}²⁴=+21.4° [c=1.3, EtOH]) (20.0 g, 147 mmol, 1.0 equiv) in MeOH (150 mL) was ozonized at −78° C. until the color of the solution changed to light blue. Then reaction mixture was purged with nitrogen and triphenylphosphine (47.4 g, 181 mmol, 1.2 equiv) was added slowly at 0° C. in several portions. After stirring for 1 h at 23° C., the reaction mixture was concentrated under reduced pressure. The crude was diluted with heptane/MTBE (3:1), mixed and the formed precipitate was filtered off. The filtrated was concentrated under reduced pressure and purified by column chromatography to afford 2-((1R,3S)-2,2-dimethyl-3-(2-oxopropyl)cyclopropyl)acetaldehyde (19.6 g, 79% yield) as a colorless liquid.

¹H NMR (400 MHz, CDCl₃, δ/ppm): 9.73 (t, J=1.8 Hz, 1H), 2.39-2.21 (m, 4H), 2.12 (s, 3H), 1.10 (s, 3H), 0.97-0.90 (m, 2H), 0.87 (s, 3H).

¹³C NMR (101 MHz, CDCl₃, δ/ppm): 208.1, 201.7, 39.5, 39.2, 29.5, 28.4, 21.1, 19.2, 17.0, 15.0.

GC/MS (EI): m/z (%): 153 (1, [M]^{+·}—·CH3), 139 (12), 111 (19), 82 (11), 81 (14), 79 (12), 67 (13), 55 (27), 43 (100), 41 (20), 39 (12). [α]_{D}²⁴=−17.9° [c=0.99, EtOH].

IR (neat, v/cm⁻¹): 2946, 2868, 1713s, 1454, 1413, 1357, 1321, 1302, 1234, 1165, 1030, 967, 921, 539.

HRMS (ESI): m/z calcd for C₁₀H₁₇O₂[M+H]⁺: 169.1223, found: 169.1226.

### b) Synthesis of 1-((1S,3R)-3-(but-2-en-1-yl)-2,2-dimethylcyclopropyl)propan-2-one

To a solution of ethyltriphenylphosphonium bromide (38.8 g, 105 mmol, 1.1 equiv) in THF (160 mL) was added at 0° C. KOtBu (11.7 g, 105 mmol, 1.1 equiv) in portions and the reaction mixture was stirred for 30 min at 0° C. The deprotonated Wittig salt mixture was then added dropwise at 0° C. to a solution of 2-((1R,3S)-2,2-dimethyl-3-(2-oxopropyl)cyclopropyl)acetaldehyde (16.0 g, 95 mmol, 1.0 equiv) in THF (160 mL). After stirring for 1 h at 0° C. and for 14 hours at room temperature, the reaction mixture was quenched with water and extracted with MTBE. The combined organic extracts were washed with brine, dried over MgSO₄ and concentrated under reduced pressure. The crude was purified by column chromatography to give 1-((1S,3R)-3-(but-2-en-1-yl)-2,2-dimethylcyclopropyl)propan-2-one (7.80 g, 46% yield, mixture of Z/E isomers in a 88:12 ratio) as a colorless liquid.

¹H NMR (400 MHz, CDCl₃, δ/ppm, mixture of isomers): 5.49-5.26 (m, 2H), 2.43-2.25 (m, 2H), 2.15 (s, 3H), 1.98-1.88 (m, 2H), 1.64-1.55 (m, 3H), 1.07 (s, 3H), 0.92 (s, 3H), 0.86-0.75 (m, 1H), 0.62 (td, J=7.5, 9.0 Hz, 1H).

¹³C NMR (101 MHz, CDCl₃, δ/ppm, Z isomer): 209.1, 129.8, 123.5, 39.4, 29.5, 28.9, 26.1, 22.0, 21.2, 17.0, 14.9, 12.8. Characteristic signals for the minor E isomer: ¹³C NMR (101 MHz, δ/ppm): 130.4, 124.6, 25.5.

GC/MS (EI), major Z isomer: m/z (%): 180 (0, [M]^{+·}), 123 (29), 107 (22), 95 (22), 81 (25), 79 (12), 69 (17), 67 (16), 55 (21), 43 (100), 41 (17).

GC/MS (EI), minor E isomer: m/z (%): 165 (5, [M]^{+·}—·CH3), 123 (21), 107 (19), 95 (20), 81 (24), 79 (11), 69 (15), 67 (16), 55 (24), 43 (100), 41 (17).

[α]_{D}²⁴=−14.8° [c=1.00, EtOH].

IR (neat, v/cm⁻¹): 3014, 2978, 2922, 2864, 1716s, 1669, 1453, 1406, 1354, 1320, 1302, 1256, 1234, 1163, 968, 696, 590, 546.

HRMS (ESI): m/z calcd for C₁₂H₂₁O [M+H]⁺: 181.1587, found: 181.1591.

Odor description: peppery, pink peppercorn, black pepper, white pepper, green, slightly metallic.

### c) Synthesis of 1-((1S,3R)-3-butyl-2,2-dimethylcyclopropyl)propan-2-one

A suspension of 1-((1S,3R)-3-(but-2-en-1-yl)-2,2-dimethylcyclopropyl)propan-2-one (7.0 g, 38.8 mmol, 1.0 equiv) and Pd/C (5%, 0.41 g, 0.5 mol %) in EtOH (140 mL) was purged with argon (3×) and hydrogenated with an H2 baloon (1 bar) for 3 h at room temperature. The reaction mixture was filtered and concentrated under reduced pressure. The crude as purified by column chromatography to give 1-((1S,3R)-3-butyl-2,2-dimethylcyclopropyl)propan-2-one (6.80 g, 96% yield) as a colorless liquid.

¹H NMR (400 MHz, CDCl₃, δ/ppm): 2.38-2.23 (m, 2H), 2.14 (s, 3H), 1.32-1.10 (m, 6H), 1.05 (s, 3H), 0.93-0.84 (m, 6H), 0.76 (td, J=7.3, 8.9 Hz, 1H), 0.52 (ddd, J=6.6, 7.5, 8.9 Hz, 1H).

¹³C NMR (101 MHz, CDCl₃, δ/ppm): 209.4, 39.5, 32.2, 29.5, 28.9, 26.3, 24.2, 22.6, 21.3, 16.9, 14.9, 14.1.

GC/MS (EI): m/z (%): 182 (1, [M]^{+·}), 125 (82), 95 (18), 83 (49), 82 (11), 81 (12), 69 (87), 67 (15), 55 (37), 43 (100), 41 (31).

[α]_{D}²⁴=−14.2° [c=1.01, EtOH].

IR (neat, v/cm⁻¹): 2956, 2927, 2860, 1715s, 1459, 1412, 1375, 1355, 1164.

HRMS (ESI): m/z calcd for C₁₂H₂₃O [M+H]⁺: 183.1743, found: 183.1747.

Odor description: green, bark, vegetable, oily, fatty.

### d) Synthesis of 1-((1S,3R)-3-butyl-2,2-dimethylcyclopropyl)hexane-2,5-dione

To a solution of diisopropylamine (5.95 mL, 42.1 mmol, 1.6 equiv) in THF was added dropwise at −60° C. n-BuLi (24.7 mL, 39.5 mmol, 1.5 equiv, 1.6 M in hexane) and the reaction mixture was stirred for 30 min at that temperature. Then a solution of 1-((1S,3R)-3-butyl-2,2-dimethylcyclopropyl)propan-2-one (4.8 g, 26.3 mmol, 1.0 equiv) in THF (10 mL) was added dropwise at −60° C. After stirring for 30 min at −60° C., 1-chloropropan-2-one (3.1 mL, 39.5 mmol, 1.5 equiv) was added dropwise at −60° C. After stirring for 30 min at −60° C. and for 30 min at 0° C., the reaction mixture was allowed to warm to room temperature and stirred for 2 hours. After quenching with brine, the reaction mixture was extracted with MTBE (2×). The combined organic extracts were washed with brine, dried over MgSO₄ and the solvent was concentrated under reduced pressure. The crude was purified by column chromatography to afford 1-((1S,3R)-3-butyl-2,2-dimethylcyclopropyl)hexane-2,5-dione (2.7 g, 11.3 mmol, 43%) as a colorless oil.

¹H NMR (400 MHz, CDCl₃, δ/ppm): 2.74-2.63 (m, 4H), 2.46-2.22 (m, 2H), 2.16 (s, 3H), 1.39-1.08 (m, 6H), 1.04 (s, 3H), 0.91-0.83 (m, 6H), 0.76 (td, J=7.2, 9.0 Hz, 1H), 0.56-0.45 (m, 1H).

¹³C NMR (101 MHz, CDCl₃, δ/ppm): 209.7, 207.2, 38.6, 36.9, 35.6, 32.2, 29.9, 28.9, 26.3, 24.2, 22.6, 21.2, 16.9, 14.9, 14.1.

GC/MS (EI): m/z (%): 238 (2, [M]^{+·}), 125 (28), 99 (100), 95 (11), 83 (16), 71 (16), 69 (52), 67 (8), 55 (22), 43 (47), 41 (19).

[α]_{D}²⁴=−14.5° [c=1.32, EtOH].

IR (neat, v/cm⁻¹): 2955, 2925, 2861, 1711s, 1458, 1401, 1357, 1180, 1162, 1095.

HRMS (ESI): m/z calcd for C₁₅H₂₇O₂[M+H]⁺: 239.2006, found: 239.2009.

### e) Synthesis of 2-((1S,3R)-3-butyl-2,2-dimethylcyclopropyl)-3-methylcyclopent-2-en-1-one

To a solution of 1-((1S,3R)-3-butyl-2,2-dimethylcyclopropyl)hexane-2,5-dione (2.70 g, 11.3 mmol, 1.1 equiv) in EtOH (60 mL) was added dropwise at room temperature aqueous 0.5 M NaOH (20 mL, 10.0 mmol, 1.0 equiv). After stirring for 24 h at 30° C., the reaction mixture was extracted with MTBE (2×). The combined organic extracts were washed with water and brine, dried over MgSO₄ and the solvent was removed under reduced pressure. The crude was purified by column chromatography and Kugelrohr distillation (130° C., 0.05 mbar) to afford 2-((1S,3R)-3-butyl-2,2-dimethylcyclopropyl)-3-methylcyclopent-2-en-1-one (compound of formula Va) (1.27 g, 52%, >99% ee) as a colorless oil.

¹H NMR (400 MHz, CDCl₃, δ/ppm): 2.49-2.44 (m, 2H), 2.38-2.31 (m, 2H), 2.06 (s, 3H), 1.63-1.49 (m, 1H), 1.41-1.21 (m, 4H), 1.18 (s, 3H), 1.02-0.95 (m, 1H), 0.88-0.82 (m, 6H), 0.82-0.68 (m, 2H).

¹³C NMR (101 MHz, CDCl₃, δ/ppm): 209.7, 174.5, 138.1, 34.7, 32.3, 31.5, 29.2, 26.9, 26.4, 22.8, 21.7, 19.0, 17.9, 16.5, 14.2.

GC/MS (EI): m/z (%): 220 (16, [M]^{+·}), 163 (54), 145 (82), 135 (44), 110 (100), 91 (61), 79 (41), 77 (41), 69 (89), 55 (60), 41 (75). [α]_{D}²⁴=+69.3° [c=1.00, EtOH].

IR (neat, v/cm⁻¹): 2955, 2928, 2860, 1701s, 1635, 1440, 1410, 1382, 1285, 1184, 1124, 1076, 997.

HRMS (ESI): m/z calcd for C₁₅H₂₅O [M+H]⁺: 221.1900, found: 221.1904.

Odor description: spicy, peppery, black pepper, woody, acet guayil, kephalis, green, rooty, vetiver fruity, jasmone.

Taste description (@3 ppb in water): woody, peppery, floral, citrus grapefruit like, slightly resinous.

### Example 2: rel-2-((1S,3R)-3-butyl-2,2-dimethylcyclopropyl)-3-methylcyclopent-2-en-1-one

### a) Synthesis of 2-(hex-1-yn-1-yl)-3-methylcyclopent-2-en-1-one

A solution of 2-iodo-3-methylcyclopent-2-en-1-one (5.00 g, 22.5 mmol, 1.0 equiv, prepared according to WO2016044558A1), copper iodide (0.39 g, 2.0 mmol, 0.09 equiv), bis(triphenylphosphine)palladium(II) dichloride (0.63 g, 0.90 mmol, 0.04 equiv) and diisopropylamine (19 mL, 0.14 mol, 6.0 equiv) in THF (120 mL) was heated to 50° C. and a solution of 1-hexyne (5.20 mL, 45 mmol, 2.0 equiv) was added dropwise over 2 hours. After stirring at 50° C. over night, the reaction mixture was diluted with water and extracted with MTBE (2×). The combined organic extracts were washed brine, dried over MgSO₄ and the solvent was removed under reduced pressure. The crude was purified by column chromatography to give 2-(hex-1-yn-1-yl)-3-methylcyclopent-2-en-1-one (3.50 g, 88%) as a colorless oil.

¹H NMR (400 MHz, CDCl₃, δ/ppm): 2.59-2.54 (m, 2H), 2.45-2.33 (m, 4H), 2.17 (s, 3H), 1.58-1.48 (m, 2H), 1.46-1.33 (m, 2H), 0.88 (t, J=7.2 Hz, 3H).

¹³C NMR (101 MHz, CDCl₃, δ/ppm): 206.1, 178.3, 126.4, 99.1, 70.7, 34.5, 31.6, 30.7, 21.8, 19.3, 18.7, 13.5.

GC/MS (EI): m/z (%): 176 (14, [M]^{+·}), 147 (28), 134 (100), 133 (55), 119 (36), 110 (27), 105 (45), 92 (49), 91 (92), 79 (25), 77 (37).

IR (neat, v/cm⁻¹): 2931, 1704s, 1621, 1430, 1381, 1339, 1293, 1198, 1112, 682, 614.

HRMS (ESI): m/z calcd for C₁₂H₁₇O [M+H]⁺: 177.1274, found: 177.1278.

### b) Synthesis of (Z)-2-(hex-1-en-1-yl)-3-methylcyclopent-2-en-1-one

In a round-bottomed flask, a suspension of 2-(hex-1-yn-1-yl)-3-methylcyclopent-2-en-1-one (2.90 g, 16.5 mmol, 1.0 equiv) and Lindlar's catalyst (50 mg) in toluene (50 mL) and pyridine (5 mL) was purged with argon and hydrogenated for 7 hours at 1 atm H₂ (balloon). After complete conversion, the reaction mixture was filtered and concentrated under reduced pressure. The crude was purified by column chromatography to give (Z)-2-(hex-1-en-1-yl)-3-methylcyclopent-2-en-1-one (2.70 g, 92%) as a colorless oil.

¹H NMR (400 MHz, CDCl₃, δ/ppm): 5.80-5.66 (m, 2H), 2.58-2.51 (m, 2H), 2.45-2.36 (m, 2H), 1.99 (s, 3H), 1.90-1.82 (m, 2H), 1.37-1.18 (m, 4H), 0.88-0.74 (m, 3H).

¹³C NMR (101 MHz, CDCl₃, δ/ppm): 208.3, 171.5, 138.0, 136.6, 118.2, 34.6, 31.7, 31.3, 29.4, 22.2, 18.4, 13.8.

GC/MS (EI): m/z (%): 178 (50, [M]^{+·}), 149 (52), 135 (50), 122 (76), 121 (47), 110 (100), 93 (54), 91 (89), 79 (80), 77 (64).

IR (neat, v/cm⁻¹): 2956, 2926, 2871, 2858, 1697s, 1625, 1438, 1406, 1377, 1284, 1173, 725.

HRMS (ESI): m/z calcd for C₁₂H₁₉O [M+H]⁺: 179.1430, found: 179.1433.

Odor description: green, jasmone, carvone, celery ketone, floral, jasmine.

### c) Synthesis of rel-2-((1S,3R)-3-butyl-2,2-dimethylcyclopropyl)-3-methylcyclopent-2-en-1-one

A suspension of 1,1′-(pyridine-2,6-diyl)bis(N-(2-(tert-butyl)phenyl)ethan-1-imine) (0.20 g, 0.47 mmol, 10 mol %, ^{2-t-Bu}PDI was prepared according to Werth, J.; Uyeda, C. Angew. Chem. Int. Ed. 2018, 57, 13902) and CoBr₂ (0.10 g, 0.47 mmol, 10 mol %) in THF (20 mL) was stirred at room temperature for 20 hours. Then, a solution of (Z)-2-(hex-1-en-1-yl)-3-methylcyclopent-2-en-1-one (0.84 g, 4.71 mmol, 1.0 equiv) in THF (15 mL) was added followed by Zn (0.62 g, 9.48 mmol, 2.0 equiv) and ZnBr₂ (1.10 g, 4.71 mmol, 1.0 equiv). The color changed slowly from yellow to violette. After stirring for 15 minutes, dichloropropane (0.98 mL, 9.48 mmol, 2.0 equiv) was added dropwise and stirring was continued at room temperature for 16 h (10% conversion, 5% product peak by GC, 85% starting material). The reaction mixture was filtered over silica and concentrated under reduced pressure. The crude was purified twice by column chromatography to afford rel-2-((1S,3R)-3-butyl-2,2-dimethylcyclopropyl)-3-methylcyclopent-2-en-1-one (16 mg, 83% purity along with 17% of starting material, <2% yield) as a colorless oil. The analytical data were identical to those reported for 2-((1S,3R)-3-butyl-2,2-dimethylcyclopropyl)-3-methylcyclopent-2-en-1-one (example 1).

Odour description for rel-2-((1S,3R)-3-butyl-2,2-dimethylcyclopropyl)-3-methylcyclopent-2-en-1-one: green (rooty, vetiver, slightly metallic) fruity (jasmone) spicy(peppery) woody (cedarwood bark, acet guayil)

### Example 3: Separation of 2-((1R,3S)-3-butyl-2,2-dimethylcyclopropyl)-3-methylcyclopent-2-en-1-one from rel-2-((1S,3R)-3-butyl-2,2-dimethylcyclopropyl)-3-methylcyclopent-2-en-1-one by Chiral Prep HPLC

A hexane solution (10 mg/mL solvent) of rel-2-((1S,3R)-3-butyl-2,2-dimethylcyclopropyl)-3-methylcyclopent-2-en-1-one (prepared as described in example 2) was separated by chiral prep HPLC using a Regis (R,R)-Whelk-O 1 column (21 injections, 20 μL per injections). A total volume of 15 mL was collected and concentrated using a nitrogen flow to ca. 1.5 mL of solvent to afford 2-((1R,3S)-3-butyl-2,2-dimethylcyclopropyl)-3-methylcyclopent-2-en-1-one with 98.2% purity (>99% ee).

[α]_{D}²⁴=minus sign according to optical rotation (OR) HPLC detector.

Odor description: woody, wood fiber, cedarwood, peppery, black pepper, green, rooty, vetiver root, slightly dusty, earthy.

### Example 4: 2-(2-butylcyclopropyl)-3-methylcyclopent-2-en-1-one

To freshly distilled CH₂Cl₂ (20 mL) was added diethylzinc (416 mg, 0.43 mL, 3.37 mmol, 2.0 equiv) under N₂. The solution was cooled in an ice bath and a solution of trifluoroacetic acid (384 mg, 0.26 mL, 3.37 mmol, 2.0 equiv) in CH₂Cl₂ (5 mL) was added dropwise very slowly to the reaction mixture via syringe. After stirring for 20 min, a solution of methylene iodide (901 mg, 0.27 mL, 3.37 mmol, 2.0 equiv) in CH₂Cl₂ (5 mL) was added. After an additional 20 min of stirring, a solution of the (E)-2-(hex-1-en-1-yl)-3-methylcyclopent-2-en-1-one (0.30 g, 1.68 mmol, 1.0 equiv) (CAS: 137097-83-7, prepared according to Tetrahedron Lett. 1991, 32, 4453) in CH₂Cl₂ (5 mL) was added, and the ice bath was removed. After stirring ofr 30 min, the reaction mixture was quenched with 0.1 N HCl (50 mL) and hexanes (25 mL), and the layers were separated. The aqueous layer was extracted with hexanes. The combined organic layers were washed with saturated NaHCO₃, H₂O, and brine and dried (Na₂SO₄), filtered and concentrated. The crude was purified by column chromatography (hexanes:MTBE=50:1) to afford 2-(2-butylcyclopropyl)-3-methylcyclopent-2-en-1-one (100 mg, 31%).

¹H NMR (400 MHz, CDCl₃, δ/ppm): 2.48-2.38 (m, 2H), 2.33-2.25 (m, 2H), 2.09 (s, 3H), 1.50-1.22 (m, 7H), 1.20-1.06 (m, 2H), 0.89 (t, J=7.1 Hz, 3H), 0.49-0.45 (m, 1H).

¹³C NMR (400 MHz, CDCl₃, δ/ppm): 209.0, 169.6, 139.1, 34.6, 34.3, 31.5, 31.2, 22.5, 17.9, 17.2, 14.5, 14.1, 11.5.

GC/MS (EI): m/z (%): 192 (40) [M+], 177 (20), 163 (18), 149 (45), 135 (45), 122 (98), 110 (65), 91 (70), 79 (100), 65 (28), 55 (40).

Odor description: green, jasmone, jasmin, spicy, anisic, celery, fenugreek.

### Example 5: rel-2-((1S,3S)-3-butyl-2,2-dimethylcyclopropyl)-3-methylcyclopent-2-en-1-one

A suspension of 1,1′-(pyridine-2,6-diyl)bis(N-(2-(tert-butyl)phenyl)ethan-1-imine) (0.24 g, 0.56 mmol, 10 mol %, ^{2-t-Bu}PDI was prepared according to Werth, J.; Uyeda, C. Angew. Chem. Int. Ed. 2018, 57, 13902) and CoBr₂ (0.12 g, 0.56 mmol, 10 mol %) in THF (5 mL) was stirred at room temperature for 20 hours (green suspension). Then, a solution of (E)-2-(hex-1-en-1-yl)-3-methylcyclopent-2-en-1-one (1.0 g, 5.61 mmol, 1.0 equiv, prepared according to Pecunioso, A.; Menicagli, R. J. Org. Chem. 1988, 53, 11, 2614) in THF (8 mL) was added followed by Zn (0.73 g, 11.2 mmol, 2.0 equiv) and ZnBr₂ (1.30 g, 5.77 mmol, 1.0 equiv). After stirring for 15 minutes, dichloropropane (1.2 mL, 11.5 mmol, 2.0 equiv) was added dropwise. After stirring for 16 hours at room temperature (29% conversion, 26% product peak by GC, 71% starting material), the reaction mixture was filtered over silica and concentrated under reduced pressure. The crude was purified three times by column chromatography to afford an enriched fraction of rel-2-((1S,3S)-3-butyl-2,2-dimethylcyclopropyl)-3-methylcyclopent-2-en-1-one (90 mg, 95% purity along with 5% starting material, 8% yield) as a yellow oil.

¹H NMR (400 MHz, CDCl₃, δ/ppm): 2.49-2.42 (m, 2H), 2.34-2.27 (m, 2H), 2.06 (s, 2H), 1.48-1.28 (m, 6H), 1.16 (s, 3H), 1.12-1.07 (m, 1H), 0.94-0.85 (m, 3H), 0.78 (s, 3H), 0.76-0.70 (m, 1H).

¹³C NMR (101 MHz, CDCl₃, δ/ppm): 209.8, 172.4, 138.4, 34.6, 32.2, 31.4, 29.6, 28.6, 25.7, 22.9, 22.7, 21.0, 21.0, 17.8, 14.1.

GC/MS (EI): m/z (%): 220 (20, [M]^{+·}), 163 (55), 145 (84), 110 (100), 91 (65), 77 (45), 69 (93), 55 (50), 41 (85).

IR (neat, v/cm⁻¹): 2956, 2921, 2858, 1697s, 1637, 1457, 1441, 1379, 1119, 1083.

HRMS (EI): m/z calcd for C₁₅H₂₄O [M]^{+·}: 220.1822, found: 220.1821.

Odor description: warm wood, patchouli, spicy, peppery, freshly grinded pepper, green, metallic, rooty, fruity, jasmone.

### Example 6: Separation of (+)-trans-2-(3-butyl-2,2-dimethylcyclopropyl)-3-methylcyclopent-2-en-1-one by prep. HPLC

A hexane solution (10 mg/mL solvent) of rel-2-((1S,3S)-3-butyl-2,2-dimethylcyclopropyl)-3-methylcyclopent-2-en-1-one (prepared as described in example 5) was separated by chiral prepHPLC using a Regis (R,R)-Whelk-O 1 column (2 μL per injection). The collected fractions were concentrated using a nitrogen flow to a 1500 ng/μL solution of (+)-trans-2-(3-butyl-2,2-dimethylcyclopropyl)-3-methylcyclopent-2-en-1-one with 99.9% purity (>99% ee).

[α]_{D}²⁴=plus sign according to optical rotation (OR) HPLC detector.

Odor description: woody, spicy, peppery, warm, round, soft.

Chiral GC (Macherey-Nagel, Hydrodex-beta-3P, 2 min@50° C.-1° C./min-2 min@230° C., H₂): t_{R}=92.7 min.; chiral HPLC (Regis (R,R)-Whelk-O 1 column, isocratic elution with n-hexane/iPrOH 90:10, 1 ml/min, 30° C., DAD @220 nm, 250 nm & 280 nm and ORD detector): t_{R}=4.97 min.

### Example 7: Separation of (−)-trans-2-(3-butyl-2,2-dimethylcyclopropyl)-3-methylcyclopent-2-en-1-one by prep. HPLC

A hexane solution (10 mg/mL solvent) of rel-2-((1S,3S)-3-butyl-2,2-dimethylcyclopropyl)-3-methylcyclopent-2-en-1-one (prepared as described in example 5) was separated by chiral prepHPLC using a Regis (R,R)-Whelk-O 1 column (2 μL per injection). The collected fractions were concentrated using a nitrogen flow to a 1000 ng/μL solution of (−)-trans-2-(3-butyl-2,2-dimethylcyclopropyl)-3-methylcyclopent-2-en-1-one with 99.9% purity (>99% ee). [α]_{D}²⁴=minus sign according to optical rotation (OR) HPLC detector.

Odor description: woody, spicy, peppery), green, metallic, rooty, ground pepper.

Chiral GC (Macherey-Nagel, Hydrodex-beta-3P, 2 min@50° C.-1° C./min-2 min@230° C., H₂): t_{R}=93.1 min.; chiral HPLC (Regis (R,R)-Whelk-O 1 column, isocratic elution with n-hexane/iPrOH 90:10, 1 ml/min, 30° C., DAD @220 nm, 250 nm & 280 nm and ORD detector): t_{R}=5.58 min.

### Example 8: 2-((1S,3R)-3-(but-2-en-1-yl)-2,2-dimethylcyclopropyl)-3-methylcyclopent-2-en-1-one

### a) 1-((1S,3R)-3-(but-2-en-1-yl)-2,2-dimethylcyclopropyl)hexane-2,5-dione

To a solution of diisopropylamine (6.4 mL, 45 mmol, 1.4 equiv) in THF (10 mL) was added dropwise at −60° C. n-BuLi (26 mL, 45 mmol, 1.3 equiv, 1.6 M in hexane) and the reaction mixture was stirred for 30 min at that temperature. Then a solution of 1-((1S,3R)-3-(but-2-en-1-yl)-2,2-dimethylcyclopropyl)propan-2-one (5.8 g, 32 mmol, 1.0 equiv, prepared according to example 1 b) in THF (10 mL) was added dropwise at −60° C. After stirring for 30 min at −60° C., 1-chloropropan-2-one (3.3 mL, 42 mmol, 1.3 equiv) was added dropwise at −60° C. After stirring for 30 min at −60° C. and for 30 min at 0° C., the reaction mixture was allowed to warm to room temperature and stirred for 2 hours. After quenching with brine, the reaction mixture was extracted with MTBE (2×). The combined organic extracts were washed with brine, dried over MgSO₄ and the solvent was concentrated under reduced pressure. The crude was purified by column chromatography to afford 1-((1S,3R)-3-(but-2-en-1-yl)-2,2-dimethylcyclopropyl)-hexane-2,5-dione (2.5 g, 32 mmol, 33%, mixture of Z/E isomers, 93:7 ratio) as a colorless oil.

¹H NMR (400 MHz, CDCl₃, mixture of isomers, δ/ppm): 5.46-5.27 (m, 2H), 2.75-2.63 (m, 4H), 2.47-2.31 (m, 2H), 2.17 (s, 3H), 1.95-1.85 (m, 2H), 1.63-1.55 (m, 3H), 1.07-1.04 (m, 3H), 0.91-0.86 (m, 3H), 0.85-0.75 (m, 1H), 0.60 (td, J=7.6, 9.0 Hz, 1H).

¹³C NMR (101 MHz, CDCl₃, major Z isomer δ/ppm): 209.6, 207.2, 129.8, 123.5, 38.5, 36.8, 35.5, 29.9, 28.8, 26.0, 22.0, 21.0, 17.0, 14.9, 12.8.

GC/MS (EI, major Z isomer): m/z (%): 236 (1, [M]^{+·}), 151 (9), 123 (22), 107 (13), 99 (100), 81 (18), 71 (15), 55 (20), 43 (56), 41 (16).

[α]_{D}²⁴=−10.0° [c=1.15, EtOH].

IR (neat, v/cm⁻¹): 2919, 1710s, 1402, 1364, 1096, 970, 694, 586.

HRMS (EI): m/z calcd for C₁₅H₂₄O₂[M]^{+·}: 236.1771, found: 236.1771.

Odor description: metallic, hot iron, slightly peppery.

### b) 2-((1S,3R)-3-(but-2-en-1-yl)-2,2-dimethylcyclopropyl)-3-methylcyclopent-2-en-1-one

To a solution of 1-((1S,3R)-3-(but-2-en-1-yl)-2,2-dimethylcyclopropyl)hexane-2,5-dione (2.2 g, 9.3 mmol, 1.2 equiv) in EtOH (25 mL) was added dropwise at room temperature aqueous 0.5 M NaOH (16 mL, 8.0 mmol, 1.0 equiv). After stirring for 16 h at 50° C., the reaction mixture was extracted with MTBE (2×). The combined organic extracts were washed with water and brine, dried over MgSO₄ and the solvent was removed under reduced pressure. The crude was purified by column chromatography and Kugelrohr distillation (130° C., 0.05 mbar) to afford 2-((1S,3R)-3-(but-2-en-1-yl)-2,2-dimethylcyclopropyl)-3-methylcyclopent-2-en-1-one (1.50 g, 73%, mixture of Z/E isomers, 9:1 ratio) as a colorless solid.

¹H NMR (400 MHz, CDCl₃, δ/ppm): 5.43-5.32 (m, 2H), 2.53-2.43 (m, 2H), 2.40-2.32 (m, 3H), 2.07-2.05 (m, 3H), 1.63-1.51 (m, 3H), 1.47-1.36 (m, 1H), 1.19-1.16 (m, 3H), 1.04-0.97 (m, 1H), 0.92-0.83 (m, 1H), 0.86 (s, 3H).

¹³C NMR (101 MHz, CDCl₃, major Z isomer, δ/ppm): 209.7, 175.1, 137.7, 130.4, 123.3, 34.7, 31.5, 29.0, 26.5, 24.2, 21.5, 18.9, 17.9, 16.6, 12.9.

GC/MS (EI, major Z isomer): m/z (%): 218 (2, [M]^{+·}), 163 (100), 145 (88), 135 (35), 105 (32), 91 (50), 79 (30), 77 (33), 55 (34), 41 (29).

[α]_{D}²⁴=+59.3° [c=1.04, EtOH].

IR (neat, v/cm⁻¹): 2915, 1699, 1635, 1439, 1381, 1289, 1123, 1009, 671.

HRMS (EI): m/z calcd for C₁₅H₂₂O [M]^{+·}: 218.1665, found: 218.1666.

Odor description: creamy, woody, aromatic, spicy, peppery, slightly green, metallic.

### Example 9: Fragrance Composition

| Ingredient | parts by weight |
|---|---|
| 2-NONENAL @1% TEC | 10 |
| AMBERMAX¹ @ 50% in DOWANOL ™ TPM | 4 |
| AMYL SALICYLATE | 80 |
| BERGAMOT OIL | 50 |
| BOISIRIS (2-ethoxy-9-methylen-2,6,6-trimethylbicyclo[3.3.1]-nonane) | 20 |
| CASHMERAN (6,7-dihydro-1,1,2,3,3-pentamethyl-4(5h)-indanone) | 5 |
| CEDARWOOD OIL | 15 |
| DAMASCONE DELTA | 3 |
| DIPROPYLENE GLYCOL | 8.1 |
| ETHYL MALTOL (3-hydroxy-2-ethyl-4h-pyran-4-one) | 2 |
| ETHYL VANILLIN (3-ethoxy-4-hydroxybenzaldehyde) | 20 |
| GALAXOLIDE S (1,3,4,6,7,8-hexahydro-4,6,6,7,8,8-hexamethylindeno[5,6-c]pyran) | 40 |
| HELIOTROPINE CRYSTALS (1,3-benzodioxole-5-carboxaldehyde) | 15 |
| ISO E SUPER (2-acetyl-1,2,3,4,5,6,7,8-octahydro-2,3,8,8-tetra-methylnaphtalene) | 250 |
| ISORALDEINE 95 ((E)-3-methyl-4-(2,6,6-trimethylcyclohex-2-en-1-yl)but-3-en-2-one) | 60 |
| JAVANOL² | 2 |
| METHYL LAITONE³ @ 10% in TEC | 20 |
| MYSTIKAL(methylundecanoic acid) @ 10% in TEC | 3 |
| OLIBANUM OIL | 50 |
| PATCHOULI OIL | 15 |
| 2-cyclohexylethyl acetate | 4 |
| NONALACTONE GAMMA (4-nonanolide) | 10 |
| RADJANOL SUPER (2-ethyl-4-(2,2,3-trimethyl-3-cyclopenten-1-yl)-2-buten-1-ol) | 15 |
| RHUBAFURAN (2,4-dimethyl-4-phenyltetrahydrofuran) | 0.5 |
| SAFRALEINE (2,3,3-trimethylindan-1-one) | 3 |
| VANILLIN | 40 |
| VETHYMINE (2,4-diethoxy-5-methylpyrimidine) @ 1% in TEC | 5 |
| VIOLET NITRILE (nona-2-6-dienenitrile) @ 10% in TEC | 0.1 |
| ZINARINE (2(2,4-dimethylcyclohex-1-yl)pyridine) | 0.3 |
| ISOPAR ™ M | 250 |
| | 1000 |

¹mixture of 2-(2,2,7,7-tetramethyltricyclo[6.2.1.0((1,6)]undec-5(4)-en-5-yl)propan-1-ol
²1-methyl-2-(1,2,2-trimethylbicyclo(3.1.0)-hex-3-ylmethyl)cyclopropyl)methanol
³8-methyl-1-oxaspiro[4.5]decan-2-one

The fragrance composition is suitable to be admixed to candles @, e.g., 4 wt %, or, for example, for room freshener with rattan sticks.

The fragrance accord represents a woody floral accord with iris and vetiver as main characters. By replacing 5 parts ISOPAR™ M with 5 parts 2-((1S,3R)-3-butyl-2,2-dimethylcyclopropyl)-3-methylcyclopent-2-en-1-one® 10% in TEC, the diffusion is clearly enhanced, and the character improves in depth and naturality, with richer orris and sandalwood facets.

### Example 10: Raspberry Flavor Composition for a Consumable Product (e.g. @ 0.06% Beverage)

| Ingredient | parts by weight |
|---|---|
| VANILLIN | 0.46 |
| RASPBERRY KETONE | 2.4 |
| ACETIC ACID | 7.0 |
| HEXENOL TRANS-2 | 0.1 |
| UNDECALACTONE DELTA | 0.1 |
| ETHYL ACETATE | 0.36 |
| ALCOHOL C 6 HEXYLIC | 0.1 |
| HEXENYL-3-CIS LACTATE | 0.18 |
| ACETALDEHYDE | 1.3 |
| IONONE BETA | 0.04 |
| STYRALLYL ALCOHOL | 0.06 |
| BUTYL-ISO PENTANOATE-ISO | 0.02 |
| METHYL-2 BUTYL BUTYRATE | 0.02 |
| FORMIC ACID | 0.5 |
| ISOAMYL ACETATE | 0.3 |
| IONONE ALPHA | 0.1 |
| HEXENOL-3-CIS | 0.34 |
| MALTOL | 0.7 |
| DIMETHYL SULFIDE | 0.02 |
| HEXYL ACETATE | 0.1 |
| HEXENYL-3-CIS ACETATE | 0.16 |
| HEXENYL-2-TRANS ACETATE | 0.04 |
| PROPYLENE GLYCOL | 985.60 |
| | 1000 |

The flavor composition above represents a typical raspberry flavor which is suitable, e.g., for a beverage. It was added at 0.06% to water comprising 1% citric acid and sweetened with 7% sugar.

By replacing 0.00008 parts DPG with 0.00008 parts 2-((1S,3R)-3-butyl-2,2-dimethylcyclopropyl)-3-methylcyclopent-2-en-1-one (resulting in a composition comprising 80 ppb of a compound of formula (I)), the flavor was perceived more natural in the profile by reducing the candy notes and generating more juice, ionon seedy notes resulting in a truer fruit-like and fresh juice character compared to the flavor composition without 2-((1S,3R)-3-butyl-2,2-dimethylcyclopropyl)-3-methylcyclopent-2-en-1-one.

1. A compound of formula (I) in the form of any one of its stereoisomers or mixture thereof 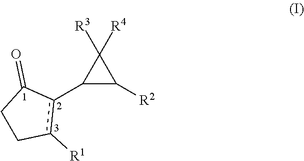
wherein
R¹ is selected from C₁-C₃ alkyl;
R² is a hydrocarbon residue comprising up to 10 carbon atoms;
R³ and R⁴ are independently selected from hydrogen and methyl with the proviso that R³ or R⁴ is methyl; and
the dotted line together with the carbon-carbon bond forms a single bond or a double bond.

2. The compound according to claim 1, wherein the relative configuration is in 1S, 3R as shown by formula (Ia) 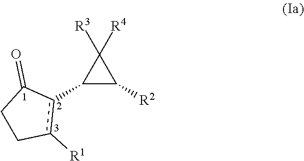
wherein
R¹ is selected from C₁-C₃ alkyl;
R² is a hydrocarbon residue comprising up to 10 carbon atoms;
R³ and R⁴ are independently selected from hydrogen and methyl with the proviso that R³ or R⁴ is methyl; and
the dotted line together with the carbon-carbon bond forms a single bond or a double bond.

3. The compound according to claim 1, wherein the hydrocarbon residue is selected from the group consisting of C₁-C₁₀ alkyl, C₂-C₁₀ alkenyl.

4. The compound according to claim 1, selected from the list consisting of
2-(3-butyl-2,2-dimethylcyclopropyl)-3-methylcyclopent-2-en-1-one,
(3-(but-2-en-1-yl)-2,2-dimethylcyclopropyl)-3-methylcyclopent-2-en-1-one,
2-(2,2-dimethyl-3-(pent-2-en-1-yl)cyclopropyl)-3-methylcyclopent-2-en-1-one,
2-(2,2-dimethyl-3-pentylcyclopropyl)-3-methylcyclopent-2-en-1-one,
2-(3-butyl-2,2-dimethylcyclopropyl)-3-propylcyclopent-2-en-1-one,
2-(3-butyl-2,2-dimethylcyclopropyl)-3-ethylcyclopent-2-en-1-one,
2-(3-(but-2-en-1-yl)-2,2-dimethylcyclopropyl)-3-ethylcyclopent-2-en-1-one,
2-(3-(hex-2-en-1-yl)-2,2-dimethylcyclopropyl)-3-methylcyclopent-2-en-1-one,
2-(3-hexyl-2,2-dimethylcyclopropyl)-3-methylcyclopent-2-en-1-one,
3-methyl-2-(2,2,3-trimethylcyclopropyl)cyclopent-2-en-1-one,
2-(3-ethyl-2,2-dimethylcyclopropyl)-3-methylcyclopent-2-en-1-one,
2-(2,2-dimethyl-3-propylcyclopropyl)-3-methylcyclopent-2-en-1-one,
2-(3-allyl-2,2-dimethylcyclopropyl)-3-methylcyclopent-2-en-1-one, and
2-(3-(but-2-en-1-yl)-2,2-dimethylcyclopropyl)-3-methylcyclopent-2-en-1-one.

5. A flavour or fragrance composition comprising a compound of formula (I) in the form of any one of its stereoisomers or mixture thereof 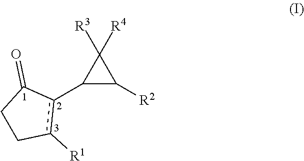
wherein
R¹ is selected from C₁-C₃ alkyl;
R² is a hydrocarbon residue comprising up to 10 carbon atoms;
R³ and R⁴ are independently selected from hydrogen and methyl with the proviso that R³ or R⁴ is methyl; and
the dotted line together with the carbon-carbon bond forms a single bond or a double bond.

6. The flavour or fragrance composition according to claim 5, wherein the compound of formula (I) is selected from the list consisting of
2-(3-butyl-2,2-dimethylcyclopropyl)-3-methylcyclopent-2-en-1-one,
(3-(but-2-en-1-yl)-2,2-dimethylcyclopropyl)-3-methylcyclopent-2-en-1-one,
2-(2,2-dimethyl-3-(pent-2-en-1-yl)cyclopropyl)-3-methylcyclopent-2-en-1-one,
2-(2,2-dimethyl-3-pentylcyclopropyl)-3-methylcyclopent-2-en-1-one,
2-(3-butyl-2,2-dimethylcyclopropyl)-3-propylcyclopent-2-en-1-one,
2-(3-butyl-2,2-dimethylcyclopropyl)-3-ethylcyclopent-2-en-1-one,
2-(3-(but-2-en-1-yl)-2,2-dimethylcyclopropyl)-3-ethylcyclopent-2-en-1-one,
2-(3-(hex-2-en-1-yl)-2,2-dimethylcyclopropyl)-3-methylcyclopent-2-en-1-one,
2-(3-hexyl-2,2-dimethylcyclopropyl)-3-methylcyclopent-2-en-1-one,
3-methyl-2-(2,2,3-trimethylcyclopropyl)cyclopent-2-en-1-one,
2-(3-ethyl-2,2-dimethylcyclopropyl)-3-methylcyclopent-2-en-1-one,
2-(2,2-dimethyl-3-propylcyclopropyl)-3-methylcyclopent-2-en-1-one,
2-(3-allyl-2,2-dimethylcyclopropyl)-3-methylcyclopent-2-en-1-one, and
2-(3-(but-2-en-1-yl)-2,2-dimethylcyclopropyl)-3-methylcyclopent-2-en-1-one.

7. A fragranced article comprising a compound of formula (I) in the form of any one of its stereoisomers or mixture thereof 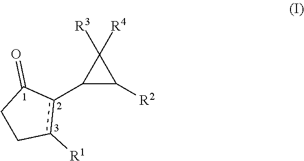
wherein
R¹ is selected from C₁-C₃ alkyl;
R² is a hydrocarbon residue comprising up to 10 carbon atoms;
R³ and R⁴ are independently selected from hydrogen and methyl with the proviso that R³ or R⁴ is methyl; and
the dotted line together with the carbon-carbon bond forms a single bond or a double bond.

8. A flavored product comprising a compound of formula (I) in the form of any one of its stereoisomers or mixture thereof 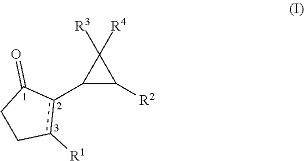
wherein
R¹ is selected from C₁-C₃ alkyl;
R² is a hydrocarbon residue comprising up to 10 carbon atoms;
R³ and R⁴ are independently selected from hydrogen and methyl with the proviso that R³ or R⁴ is methyl; and
the dotted line together with the carbon-carbon bond forms a single bond or a double bond.

9. A method of making a fragranced article or flavored product comprising admixing to a consumer product base or consumable product base an olfactory acceptable amount of a compound of formula (I) in the form of any one of its stereoisomers or mixture thereof 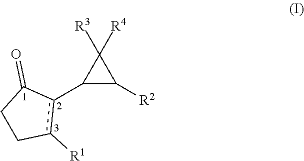
wherein
R¹ is selected from C₁-C₃ alkyl;
R² is a hydrocarbon residue comprising up to 10 carbon atoms;
R³ and R⁴ are independently selected from hydrogen and methyl with the proviso that R³ or R⁴ is methyl; and
the dotted line together with the carbon-carbon bond forms a single bond or a double bond.

10. A method of improving, enhancing and/or modifying a consumer product base or consumable product base by means of adding thereto an olfactory acceptable amount of a compound of formula (I) in the form of any one of its stereoisomers or mixture thereof 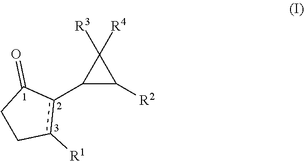
wherein
R¹ is selected from C₁-C₃ alkyl;
R² is a hydrocarbon residue comprising up to 10 carbon atoms;
R³ and R⁴ are independently selected from hydrogen and methyl with the proviso that R³ or R⁴ is methyl; and
the dotted line together with the carbon-carbon bond forms a single bond or a double bond.
